(19) United States
(12) Patent Application Publication
(10) Pub. No.: US 2021/0236844 A1
(43) Pub. Date: Aug. 5, 2021

(54) A METHOD AND DEVICE FOR REAL TIME MONITORING AND PREDICTION OF BILIRUBIN LEVELS AND ASSOCIATED NOTIFICATIONS IN NEONATES

(71) Applicant: KONINKLIJKE PHILIPS N.V., EINDHOVEN (NL)

(72) Inventors: ANSHUL JAIN, BANGALORE (IN); PRAVIN PAWAR, BANGALORE (IN); RAVINDRA BHAT, EINDHOVEN (NL); SHRUTIN ULMAN, BANGALORE (IN); RAVI BABU SUNDARAMOORYHY, BANGALORE (IN); NAGARAJU BUSSA, BANGALORE (IN)

(21) Appl. No.: 17/050,563

(22) PCT Filed: Apr. 24, 2019
(86) PCT No.: PCT/EP2019/060414

§ 371 Date: Oct. 26, 2020

Related U.S. Application Data
(60) Provisional application No. 62/663,331, filed on Apr. 27, 2018.

(51) Int. Cl. A61N 5/06

(52) U.S. Cl. CPC A61N 5/0621; A61N 2005/0663; A61N 2005/0645; A61N 2005/0627

(57) ABSTRACT

A phototherapy monitoring device (10) includes a housing (12) configured for attachment to a patient, and a user interfacing device. An optical bilimbin sensor (14) includes one or more light sources (16) operative to generate probe light and arranged on or in the housing such that the probe light is reflected from or transmitted through skin of the patient when the housing is attached to the patient; and one or more photodetectors (18) arranged on or in the housing to detect the probe light reflected from or transmitted through the skin of the patient. At least one electronic processor (28) is disposed on or in the housing and programmed to: continuously generate a current bilimbin level measurement from the detected probe light reflected from or transmitted through the skin of the patient; and control the user interfacing device to generate a notification when the current bilirubin level measurement satisfies a safe bilimbin level.

### FIELD

The following relates generally to the phototherapy arts, infant safety monitoring arts, jaundice treatment arts, patient monitoring arts, and related arts.

### BACKGROUND

Bilirubin is a yellow pigment in blood and stool resulting from the breakdown of red cells. In the liver, bilirubin is excreted into the bile duct and stored in gallbladder. Eventually, bilirubin is released into the small intestine as bile to help digest fats and ultimately excreted with the stool. Hyperbilirubinemia refers to too much bilirubin in the blood and also results in yellowing of skin, eyes and other tissues leading to conditions such as jaundice. About 60% of term newborns and 80% of premature babies develop hyperbilirubinemia which may lead to jaundice. Newborn infants often have mild jaundice due to normal changes in the metabolism of bilirubin. These changes can be first signs of a medical problem. Jaundice in a newborn can be very serious and life threatening if left untreated. Bilirubin in the infant's blood may be tested several times in the first few days to check liver functioning, which is an invasive method.

Phototherapy is the most common treatment for reducing high bilirubin levels that cause jaundice in a newborn. A baby with jaundice may need to stay under a phototherapy light for several days. Potential problems that may occur during phototherapy include skin rash, damage to the nerve layer at the back of the eye (e.g., retina), dehydration, and separation from the infant's mother. Doctors check bilirubin levels at certain interval of time. Traditional bilirubin level screening has a number of limitations such as blood is collected for testing via venepuncture, which causes the baby pain and adds additional infection risks.

Currently, there exists non-invasive devices to check bilirubin levels in infant. A ColorMate III device (available from Chromatics Color Sciences, Intl, Inc.; Rockville, Md., USA) checks infant bilirubin levels based on color of skin and estimates bilirubin from skin reflectance. This device uses a Xenon flash tube and light sensors to measure wavelengths from 400 nm to 700 nm; and requires a baseline Total Serum Bilirubin (TSB) reading on each newborn baby. A Minolta/Air Shields JM-103 device (available from Konica Minolta Sensing Americas, Inc. Ramsey, N.J., USA) determines the bilirubin from the subcutaneous tissue of neonate; and determines difference in the optical differences of reflected light at 450 nm and 550 nm by infant skin. A Philips® Bilicheck (available from Koninklijke Philips N.V., Eindhoven, the Netherlands) measures bilirubin transcutaneously by using the visible light (380-760 nm) reflected by skin and subtracts light absorption of interfering factors such as hemoglobin and melanin to obtain bilirubin concentration. This device includes a disposable tip for each measurement. A Bilicam device (University of Washington, Seattle, Wash., USA) is a smartphone based medical device that uses the embedded cellphone camera and a paper based color calibration card to estimate jaundice from which the bilirubin level is inferred. The approach uses color balance in obtained images, obtains intensities of various reflected wavelengths and chromatic and achromatic properties from the skin, and estimates a bilirubin level using machine learning techniques. A CoSense® End-Tidal Carbon Monoxide (ETCO) Monitor (available from Capnia, Inc. Redwood City, Calif., USA) automates non-invasive detection of analytes in exhaled breath. This device acquires a breath sample with a tube inserted into a nostril for about thirty seconds, and displays the results in three to four minutes.

The following discloses new and improved systems and methods to address these problems.

### SUMMARY

In one disclosed aspect, a phototherapy monitoring device includes a housing configured for attachment to a patient, and a user interfacing device. An optical bilirubin sensor includes one or more light sources operative to generate probe light and arranged on or in the housing such that the probe light is reflected from or transmitted through skin of the patient when the housing is attached to the patient; and one or more photodetectors arranged on or in the housing to detect the probe light reflected from or transmitted through the skin of the patient. At least one electronic processor is disposed on or in the housing and programmed to: continuously generate a current bilirubin level measurement from the detected probe light reflected from or transmitted through the skin of the patient; and control the user interfacing device to generate a notification when the current bilirubin level measurement satisfies a safe bilirubin level.

In another disclosed aspect, a phototherapy monitoring device includes a housing configured for attachment to a patient At least two illuminators are secured to the housing and arranged to emit light towards at least a portion of the patient. A first illuminator is configured to emit light at a first wavelength and a second illuminator being configured to emit light at a second, different wavelength. A photodetector is configured to measure intensities of light reflected from the patient at the first and second wavelengths. At least one electronic processor programmed to: continuously estimate a bilirubin level in the patient by comparing the measured intensity of light at the first and second wavelengths; and generate an indication of whether the continuously estimated bilirubin level in the patient has decreased to a safe level.

In another disclosed aspect, a method of monitoring phototherapy delivered to a patient includes: with at least two illuminators secured to a housing attached to the patient, emitting light towards at least a portion of the patient at a first wavelength and a second, different wavelength; with at least two photodetectors, measuring intensities of light reflected from the patient at the first and second wavelengths; and with at least one electronic processor: continuously estimate a bilirubin level in the patient by comparing the measured intensity of light at the first and second wavelengths; apply a linear model to the continuously estimated bilirubin level in the patient to determine a remaining amount of time for phototherapy, the model further receiving as inputs at least patient age, patient skin color, and the safe level; and generate an indication of whether the continuously estimated bilirubin level in the patient has decreased to a safe level based on the continuously estimated bilirubin level.

One advantage resides in continuous monitoring of bilirubin levels in neonates during phototherapy.

Another advantage resides in providing immediate notification when a continuously measured level of bilirubin reaches a safe level.

Another advantage resides in reduction of skin irritation, dehydration, retina damage, and hypocalcaemia in neonates undergoing phototherapy by way of rapidly determining when the phototherapy has achieved the clinical goal, and/or by estimating when the phototherapy will achieve the clinical goal.

Another advantage resides in reduction of separation time between the neonate and the mother by enabling termination of the phototherapy as soon as the clinical bilirubin level goal is achieved.

Another advantage resides in sending automatic notifications when a measured level of bilirubin reaches a safe level.

Another advantage resides in determining a correct exposure time of the neonate to phototherapy.

Another advantage resides in determining a remaining time for a neonate to undergo phototherapy.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically shows a device for monitoring phototherapy to a patient according to one aspect.

FIG. 2 diagrammatically shows another view of the device of FIG. 1.

FIG. 3 diagrammatically shows another view of the device of FIG. 1.

FIG. 4 diagrammatically a model used by the device of FIG. 1.

FIG. 5 diagrammatically shows an operational flow chart for operation of the device of FIG. 1.

### DETAILED DESCRIPTION

Hyperbilirubinemia is a condition in which the blood contains too much bilirubin. Jaundice is a chief symptom of hyperbilirubinemia, and the condition is common in newborn infants due to delayed development of bilirubin removal functionality of the liver. Phototherapy is a common treatment for hyperbilirubinemia, but the therapy has various adverse side effects and hence is preferably applied only for a sufficient time to reduce the bilirubin to safe levels. Conventionally, periodic blood tests or a handheld bilirubin meter is used to monitor the bilirubin level intermittently.

The following discloses an automated bilirubin meter that straps to the infant, e.g. to the forehead or belly, and provide continuous measurement of bilirubin level. The bilirubin meter is sized to minimally cover a portion of the infant, and does not affect phototherapy light from reaching the infant. Phototherapy light reflected by the infant's skin has a low intensity and is sensitive to ambient light. Hence, a case or housing of the device is made from an opaque material. The opaque structure of the device prevents ambient light interference in the interaction of blue and green light emitted from LEDs of the device with the infant's skin.

Continuous bilirubin measurement provides enhanced capabilities over a conventional handheld bilirubin meter. The bilirubin level can be tracked over time, and the bilirubin level versus time curve can be analyzed to estimate when the phototherapy will reduce the bilirubin to a safe level. In embodiments disclosed herein, a suitable model is a linear model parameterized by infant age, skin color (i.e. dark or light, not the jaundice-induced yellowing), initial (or current) bilirubin level, and the target bilirubin level (e.g., prescribed by a physician). More complex models, e.g. a quadratic or exponential model, are also contemplated. In existing phototherapy devices the intensity of the therapeutic blue light is fixed; however, if this intensity is an adjustable parameter then the physician might elect to increase the therapeutic blue light intensity if the estimated time-to-safe-level is deemed to be too long.

The continuous bilirubin measurement is also contemplated to provide for automated control. In one approach, the phototherapy device may be turned off when the bilirubin reaches a safe (e.g. physician-prescribed) level. To avoid premature termination of the phototherapy due to measurement noise, this may be done only after the safe level is maintained for some time interval. If the therapeutic blue light intensity is adjustable then in a more advanced embodiment the therapeutic blue light intensity may be controlled, e.g. using the linear model prediction to achieve a target time to safe level.

The continuous bilirubin measurement is preferably coupled with automatic notifications, e.g. to notify medical personnel when the bilirubin has reached the safe (e.g. physician-prescribed) level. Other embodiments disclosed herein include providing an optical detector with a bandpass filter to measure the therapeutic blue light intensity, and to provide an alert if the phototherapy device is not delivering the blue light at a therapeutically effective (or physician-prescribed) intensity level.

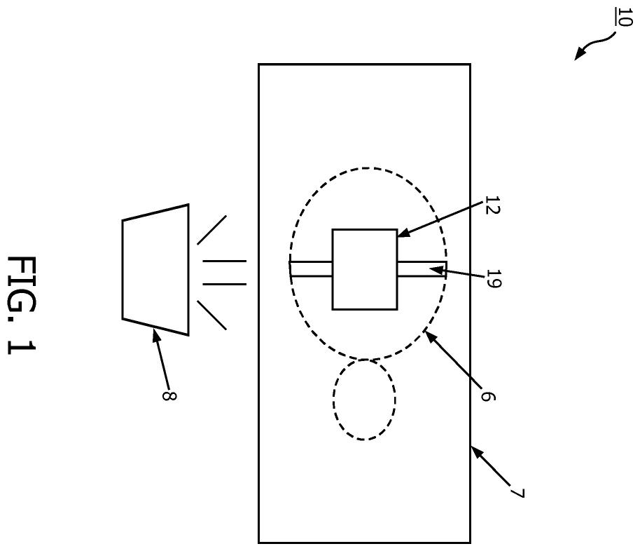

With reference to FIG. 1, a neonate 6 (also referred to herein as “infant”) disposed on or in a bed, crib, or other support 7 receives phototherapy via a phototherapy device 8 for treating hyperbilirubinemia (a high level of bilirubin in the blood; also sometimes referred to a jaundice due to a common symptom of yellowing of the skin). The phototherapy device 8 delivers therapeutic blue light that breaks bilirubin down into a form that is more easily excreted by the body. Typically, the therapeutic blue light is visible light at a wavelength of 500 nm or shorter (e.g., the blue therapeutic light may be blue-green light, and/or may have some violet component or so forth) or having a substantial spectral component at 500 nm or shorter. The illustrative phototherapy device 8 is shown diagrammatically; more typically, the phototherapy device may include two or more lamps emitting therapeutic blue light from different angles to maximize the area of skin of the infant 6 that is illuminated by the therapeutic light. In some contemplated embodiments, the phototherapy device 8 may be a phototherapy garment that includes blue LEDs or the like embedded in the garment and designed to deliver the therapeutic blue light for the phototherapy. This arrangement has certain benefits, such as illuminating a greater fraction of the infant's skin than can generally be achieved using overhead or side-mounted blue lamps.

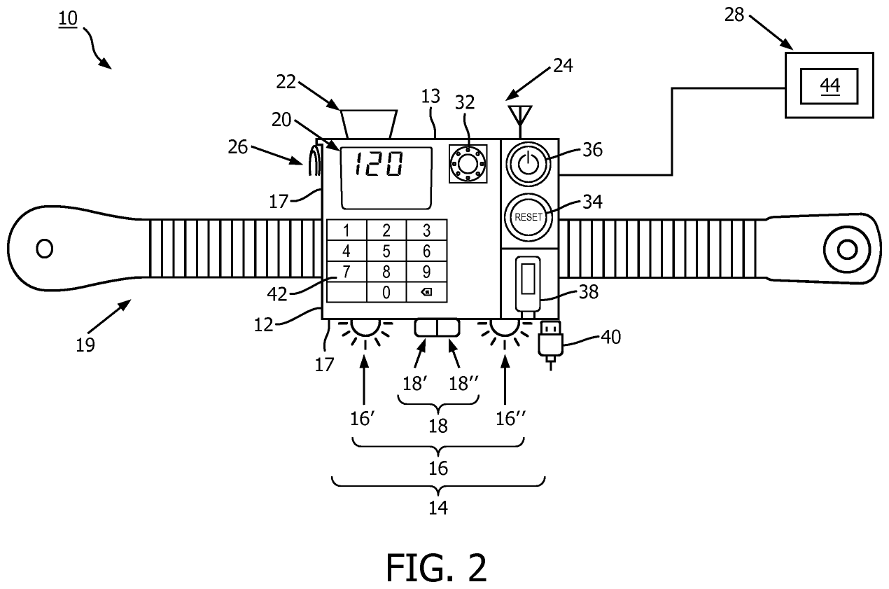

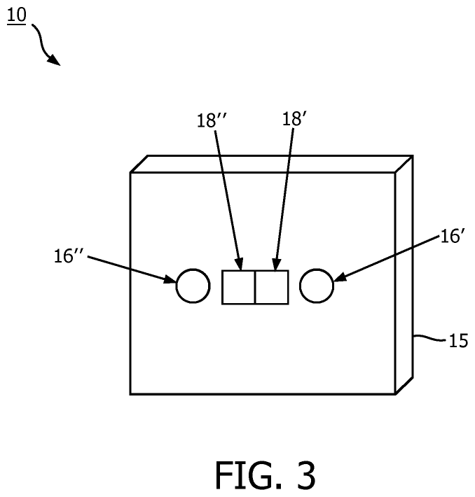

During the phototherapy, the level of bilirubin in the blood is continuously monitored by an illustrative phototherapy monitoring device 10. In particular, the illustrative phototherapy monitoring device 10 is configured for used with the neonate 6, but more generally may be used for any patient (infant or adult) afflicted with hyperbilirubinemia and/or exhibiting jaundice. With reference to FIGS. 2 and 3, and with continuing reference to FIG. 1, the device 10 includes a housing 12 configured for attachment to the patient. In some examples, the housing 12 can be box-shaped, and include a top face 13, a bottom face 15 (FIG. 3), and a plurality (i.e., 4) side faces 17 disposed therebetween. The housing 12 can also have any other suitable shape (e.g., disk-shaped and so forth). In one example, the housing 12 is attached to the patient with a belt 19 configured (e.g., sized and shaped) to be wrapped around the chest (as shown) or the forehead of the patient. In other examples, the housing 12 can be attached to the patient with any other suitable device, such as an adhesive, or a phototherapy garment. The phototherapy garment can be infant blanket or garment configured (e.g. sized and shaped) to be worn by the patient (i.e., an infant in the case of an infant phototherapy device). In one approach, the phototherapy garment may include an elastic gather, elastic band, or the like to provide pressure to hold the housing 12 against the skin of the patient.

With continuing references to FIGS. 1 and 2, and with continuing reference to FIG. 1, the phototherapy monitoring device 10 includes an optical bilirubin sensor 14 configured measure bilirubin levels of the patient. The bilirubin sensor 14 includes one or more light sources or illuminators 16 disposed on or otherwise secured to a portion of the housing 12. In some embodiments, the light sources 16 comprise light emitting diodes (LEDs) operative to generate or emit probe light towards at least a portion of the patient such that the probe light is reflected from or transmitted through skin of the patient when the housing is attached to the patient. An issue with providing continuous monitoring is that if the phototherapy monitoring device 10 occludes the therapeutic blue light at the monitored patch of skin, then the measured bilirubin level in that occluded skin patch may not accurately reflect the average bilirubin level in skin tissue generally. (In this regard, it is noted that the probe light emitted by the LEDs 16 of the bilirubin sensor 14 may be of too low intensity and/or of too long wavelength to be therapeutically effective for breaking down bilirubin. Said another way, the LEDs 16 of the bilirubin sensor 14 typically do not deliver therapeutic light.)

The bilirubin sensor 14 also includes one or more photodetectors or receivers 18 configured to measure probe light reflected from the patient. As shown in FIG. 2, the light sources 16 includes a first light source 16′ configured to emit or generate light at a first wavelength (e.g., 550 nm or green light), and a second light source 16″ configured to emit or generate light at a second wavelength that is different from the first wavelength (e.g., 450 nm or blue light). The photodetectors 18 are configured to measure intensities of the reflected light at the first and second wavelengths. The photodetectors 18 are arranged on or in the housing 12 to detect the probe light reflected from or transmitted through the skin of the patient. As shown in FIG. 1, the photodetectors 18 include a first photodetector 18′ configured to detect light reflected at the first wavelength, and a second photodetector 18″ configured to detect light reflected at the second wavelength. To achieve wavelength-selective detection, a bandpass filter may for example be mounted at the light entry aperture of the photodetector 18. In another example, the light sources 16 are configured or operative to generate polychromatic probe light. The photodetectors 18 are configured or operative to detect a spectrum of the polychromatic probe light reflected from the skin of the patient.

The light sources 16 and the photodetectors 18 are arranged on the bottom face 15 of the housing 12 (The device 10 shown in FIG. 2 is arranged so that the light sources and photodetectors are visible for convenience). The light sources 16 and the photodetectors 18 are shown in FIG. 3 as being disposed on the bottom face 15 of the housing 12. Advantageously, ambient light is blocked from reaching the light sources 16 and the photodetectors 18 to prevent interference of ambient light being detected by the photodetectors. In other words, the photodetectors 18 are arranged to only detect light reflected from skin originating from the light sources 16.

As shown in FIG. 2, the phototherapy monitoring device 10 also includes a user interfacing device. For example, the user interfacing device comprises one or more of: (1) a display screen 20 disposed or mounted on the housing 12; (2) a loudspeaker 22 disposed on or in the housing; (3) a wireless communication interface 24 (e.g., an Internet of Things antenna) disposed on or in the housing; and/or (4) an alert light 26 disposed on a portion of the housing. The user interfacing device is configured for user interaction with a medical professional (e.g., a doctor, a nurse, a technician, and so forth) when an indication is generated based on the reflected light measured by the photodetector(s) 18.

The device 10 further includes a control circuit that is operatively connected to the illuminators 16 and the receivers 18, and disposed on or in the housing 12. The control circuit may, for example, comprise at least one electronic processor 28, a microprocessor or microcontroller and ancillary electronic components such as a memory chip (e.g. EPROM, EEPROM, flash memory, et cetera), discrete components (e.g. resistors, capacitors), and/or so forth, with (for example) the memory chip storing executable code (e.g. software or firmware) executable by the microprocessor or microcontroller to perform processing functions as described herein. Optionally, the control circuit may additionally or alternatively include analog processing circuitry, e.g. an operational amplifier (op-amp) circuit designed to compare inputs including a reflected light intensity measurement reading from the photodetectors 18 and a reference signal corresponding to the maximum permissible intensity measurement and to generate a control signal based on the comparison.

In some embodiments, the processor 28 is programmed to continuously generate or estimate a current bilirubin level measurement in the patient from the detected probe light reflected from or transmitted through the skin of the patient. To do so, the the processor 28 is programmed to continuously compare the measured intensity of light at the first and second wavelengths detected by the photodetectors 18. This operation can performed using known methods (see, e.g., Penhaker et al., “Advanced Bilirubin Measurement by a Photometric Method,” ELEKTRONIKA IT ELECTROTECHNIKA, ISSN 1392-1215, Vol. 19, No. 3, 2013). In one example, the electronic processor 28 is programmed to continuously generate the current bilirubin level measurement from the intensities of the detected first and second probe light reflected from the skin of the patient. In another example, the electronic processor 28 is programmed to continuously generate the current bilirubin level measurement from skin color data derived from the detected spectrum of the polychromatic probe light reflected from the skin of the patient.

In other embodiments, accuracy of skin-based bilirubin measurement are lower than a blood-based measurement due to the presence of skin pigmentations, such as melamine. In some embodiments, the electronic processor 28 is programmed to calibrate the photodetectors 18 with bilirubin levels obtained from blood of the patient (this is typically done at least once a day immediately after birth in the hospital). These bilirubin levels are entered in the device 10. After one or more such values are entered, the device 10 can compensates for the infant's individual skin pigmentation and read bilirubin accurately.

In some embodiments, the processor 28 is programmed to apply a model 44 (see FIG. 3) to the current bilirubin level measurement or the continuously estimated bilirubin level to estimate a time at which the safe bilirubin level will be reached. For example, the model 44 may be a linear model receiving as inputs at least patient age, patient skin color, and the safe bilirubin level of the patient. In another example, the processor 28 is programmed to apply the model 44 (e.g., a linear model) to the continuously estimated bilirubin level in the patient to determine a remaining amount of time for phototherapy, the model further receiving as inputs at least patient age, patient skin color, and the safe level.

The processor 28 is also programmed to control the user interfacing device to generate a notification or indication when the current bilirubin level measurement satisfies a safe bilirubin level, such as when the continuously estimated bilirubin level in the patient has decreased to a safe level. In some embodiments, the processor 28 is programmed to control the display screen 20 to output display a textual or visual message of the indication that the current bilirubin level measurement satisfies a safe bilirubin level. The display screen 20 can also be controlled to output or display the continuously estimated current bilirubin level as, for example, a real-time value and/or a trend line. In another example, the processor 28 is programmed to control the loudspeaker 22 to sound an audible alarm when the current bilirubin level measurement satisfies the safe bilirubin level. In another example, the processor 28 is programmed to control the wireless communication interface 24 to transmit the notification as an electronic message to the medical professional. In another example, the processor 28 is programmed to control the alert light 26 to illuminate to output the generated indication when the current bilirubin level measurement satisfies a safe bilirubin level.

Referring back to FIG. 1, in some embodiments the processor 28 is programmed to control the phototherapy device 8 to stop applying or emitting phototherapy (e.g., blue) light when the current bilirubin level measurement satisfies the safe bilirubin level, such as when the estimated bilirubin level has decreased to the safe level. In addition, a high rate of fall of bilirubin is also not healthy for the neonates. Phototherapy light breaks up bilirubin molecules and the bi-products are known to cause oxidative stress. The processor 28 is programmed to control the phototherapy device 8 to adjust a rate of phototherapy light emitted towards the patient by varying the phototherapy intensity (or switching off) phototherapy lights of the phototherapy device 10.

In the foregoing examples, the determination of when the bilirubin level reaches the safe level may be done in such a way so as to limit the effects of measurement noise. For example, the safe level may be determined to have been reached only when the continuously measured bilirubin level remains at or below the safe level for some pre-set time interval, e.g. for at least one hour. It is also noted that “continuous” measurement of the bilirubin level encompasses digital sampling at reasonably fast sampling rates, e.g. a digital bilirubin measurement value may updated every second, or every minute, or every two minutes, or so forth. By “continuous”, it is meant that the bilirubin measurements are acquired sequentially in an automated fashion, as opposed to, for example, a manual bilirubin meter that would need to be positioned manually and triggered to acquire a bilirubin measurement.

Referring back to FIG. 2, the device 10 includes other various optional components. For example, the illustrative device 10 includes a photosensor 32 configured to measure light intensity at the wavelength of the blue phototherapy light emitted from the phototherapy device 8. The device may be programmed to issue a notification if the phototherapy light intensity is below some minimum threshold, or if the desired phototherapy light intensity is known a priori or input into the device, e.g. via a keyboard 42, then the notification may be issued if the measured therapeutic light intensity is different by more than a preset amount from the desired intensity. A reset button 34 is used to erase existing data and reset the device 10. A power-off button 36 is used to turn the device 10 on and off. (In a variant embodiment the power may be cycled off/on to reset the device, eliminating the need for the reset button). A battery 38 allows the device 10 to operate without being plugged in to an electrical outlet. A charging port 40 is used to charge the battery 38. Alternatively, in a wired design electrical power may be supplied by a power cord; or, if the phototherapy monitoring device is integrated into a phototherapy garment then electrical power may be supplied via wires embedded in the garment. The keyboard 42 is used to input data to the device 10. In a variant embodiment, if the wireless communication interface 24 is provided then it is contemplated to provide an application program (“app”) that can be downloaded and run on a cellular telephone (cellphone), tablet computer, or the like, which app communicates with the phototherapy monitoring device 10 via the wireless communication interface 24 (e.g. a Bluetooth and/or WiFi interface) and provides a user interface via which the user may configure the phototherapy monitoring device 10 and optionally otherwise interact with the device 10 (e.g., calibrate the device 10 when it placed on the neonate, receive bilirubin level readings, notifications, et cetera). The wireless communication interface 24 also enables connectivity to a hospital network (not shown) for these purposes.

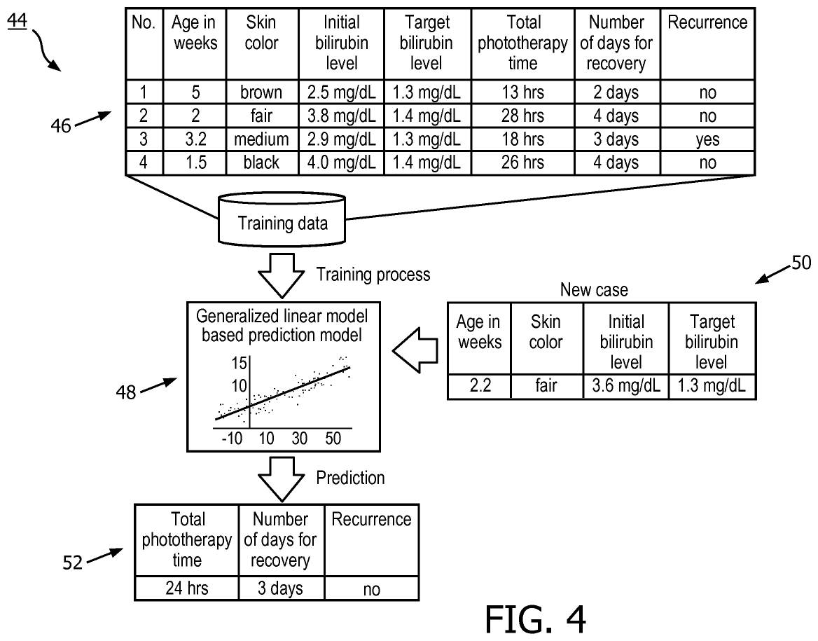

With reference to FIG. 4, an example of the model 44 implemented by the electronic processor 28 is shown. The model can employ a machine learning technique such as Generalized Linear Model (GLM) for individualized prediction of the phototherapy time likely to be required to achieve the designated safe bilirubin level. The GLM model is used for a continuous response variable given continuous and/or categorical predictors. The GLM model is suitable to predict number of hours in a phototherapy machine (which is a continuous variable). Training data 46 suitably consists of phototherapy details of successfully treated cases. In the illustrative implementation, relevant parameters of the training data include: 1) age of neonate in weeks, 2) skin color, 3) bilirubin level prior to the start of phototherapy treatment, 4) safe bilirubin level, 5) total phototherapy time taken to reduce bilirubin level to safe level, 6) number of days for recovery, and 7) whether the jaundice has recurred after initial treatment. This data is used to train a decision tree 48 based prediction model. After successful training, when an unseen case 50 is provided as input to this model, the decision tree generates output data 52, which predicts required number of hours for phototherapy treatment, number of days for recovery and whether jaundice will recur. The inputs for applying the trained GLM to predict the time to safe bilirubin level may include patient age, patient skin color, and the safe bilirubin level. The currently measured bilirubin level is a further input to the GLM, as the GLM infers the time likely to be required for the bilirubin level to decrease from the current level to the safe level. This prediction may be variously used. For example, it may be displayed on the display 20 (and/or on a cellphone or tablet computer display if such a mobile device is running an app providing the user interfacing). This predicted time to safe bilirubin level allows for medical personnel to schedule the therapy time, determine how long the phototherapy device 8 will need to be assigned to the infant 6 receiving the phototherapy, or so forth.

In some embodiments, if the phototherapy device 8 provides for adjustment of the intensity level of the therapeutic light then this intensity may be adjusted based on the predicted time to safe bilirubin level. For example, if that time is deemed to be too long then the therapeutic light intensity may be increased to expedite the phototherapy. In these embodiments, the therapeutic blue light intensity should be another input for training of the model 44, and the therapeutic blue light intensity is also an input during the inference phase. In embodiments in which the phototherapy device 8 is controlled by the phototherapy monitoring device 10, it is also contemplated to automatically control the therapeutic blue light intensity based on the estimated time-to-safe bilirubin level. For example, the therapeutic blue light may be set to the lower of (1) the minimum therapeutic blue light intensity for achieving safe bilirubin level by a pre-set time interval or (2) a pre-set absolute maximum allowable therapeutic blue light intensity.

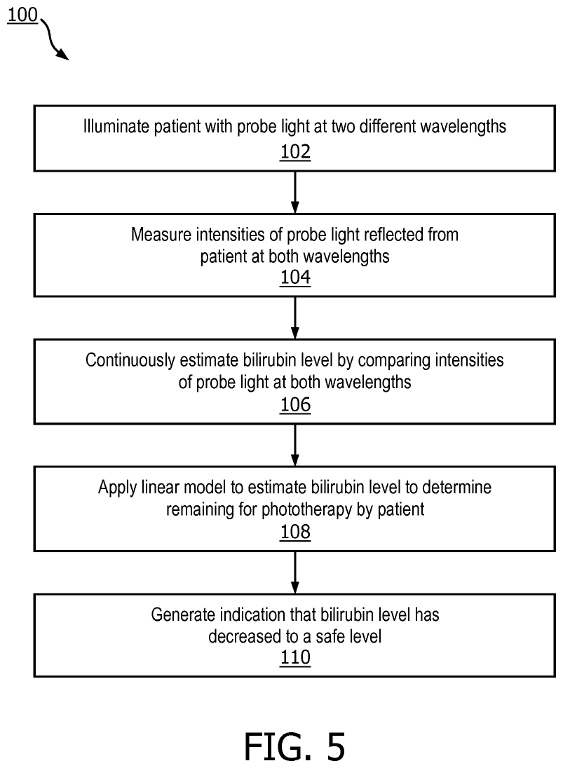

With reference to FIG. 5, operation of the device 10 is diagrammatically flowcharted as a method 100 of monitoring phototherapy delivered to the patient. At 102, probe light is emitted towards at least a portion of the patient by the first illuminator 16′ at a first wavelength and by the second illuminator 16″ a second, different wavelength. At 104, intensities of probe light reflected from the patient at the first and second wavelengths are measured with at least two corresponding photodetectors 18′ and 18″. At 106, a bilirubin level in the patient is continuously estimated with the at least one electronic processor 28 by comparing the measured intensity of probe light at the first and second wavelengths. At 108, a linear model is applied with the processor 28 to the continuously estimated bilirubin level in the patient to determine a remaining amount of time for phototherapy in which the model receives, as inputs, at least patient age, patient skin color, and a safe level. At 110, an indication of whether the continuously estimated bilirubin level in the patient has decreased to a safe level is generated with the processor 28 based on the continuously estimated bilirubin level.

The disclosure has been described with reference to the preferred embodiments. Modifications and alterations may occur to others upon reading and understanding the preceding detailed description. It is intended that the disclosure be construed as including all such modifications and alterations insofar as they come within the scope of the appended claims or the equivalents thereof.

1. A phototherapy monitoring device, comprising:
a housing configured for attachment to a patient;
a user interfacing device;
an optical bilirubin sensor including:
one or more light sources operative to generate probe light and arranged on or in the housing such that the probe light is reflected from or transmitted through skin of the patient when the housing is attached to the patient; and
one or more photodetectors arranged on or in the housing to detect the probe light reflected from or transmitted through the skin of the patient; and
at least one electronic processor A disposed on or in the housing and programmed to:
continuously generate a current bilirubin level measurement from the detected probe light reflected from or transmitted through the skin of the patient; and
control the user interfacing device to generate a notification when the current bilirubin level measurement satisfies a safe bilirubin level.

2. The phototherapy monitoring device of claim 1, wherein the at least one electronic processor is further programmed to:
control a phototherapy device to change a rate of emitted phototherapy light towards the patient when the current bilirubin level measurement satisfies the safe bilirubin level.

3. The phototherapy monitoring device claim 1, wherein the at least one electronic processor further programmed to:
apply a model to the current bilirubin level measurement to estimate a time at which the safe bilirubin level will be reached.

4. The phototherapy monitoring device of claim 3 wherein the model is a linear model further receiving as inputs at least patient age, patient skin color, and the safe bilirubin level.

5. The phototherapy monitoring device of claim 1, wherein the one or more light sources of the optical bilirubin sensor are arranged on or in the housing to generate the probe light directed outward from a periphery of the housing.

6. The phototherapy monitoring device claim 1, wherein the at least one electronic processor is further programmed to:
calibrate the one or more photodetectors with bilirubin levels obtained from blood of the patient.

7. The phototherapy monitoring device of claim 1, wherein the user interfacing device comprises one or more of:
a display mounted on the housing and displaying the notification,
a loudspeaker sounding the notification comprising an audible alarm, or
a wireless communication interface via which the notification is transmitted as an electronic message.

8. The phototherapy monitoring device of claim 1, wherein the user interfacing device comprises a display and the at least one electronic processor further programmed to
continuously display the current bilirubin level measurement as at least one of a real-time value and a trend line.

9. The phototherapy monitoring device of any one of claim 1, wherein:
the one or more light sources of the optical bilirubin sensor are operative to generate first probe light at a first wavelength and second probe light at a second wavelength different from the first wavelength;
the one or more photodetectors are arranged on or in the housing to detect respective intensities of the first and second probe light reflected from the skin of the patient; and
the at least one electronic processor is programmed to continuously generate the current bilirubin level measurement from the intensities of the detected first and second probe light reflected from the skin of the patient.

10. The phototherapy monitoring device of claim 1, wherein:
the one or more light sources of the optical bilirubin sensor are operative to generate polychromatic probe light;
the one or more photodetectors are arranged on or in the housing to detect a spectrum of the polychromatic probe light reflected from the skin of the patient; and
the at least one electronic processor is programmed to continuously generate the current bilirubin level measurement from skin color data derived from the detected spectrum of the polychromatic probe light reflected from the skin of the patient.

11. The phototherapy monitoring device of claim 1, wherein the housing is configured for attachment to a patient by at least one of a belt an adhesive, or by being embedded in a phototherapy garment.

12. A phototherapy monitoring device, comprising:
a housing configured for attachment to a patient;
at least two illuminators secured to the housing and arranged to emit light towards at least a portion of the patient, a first illuminator being configured to emit light at a first wavelength and a second illuminator being configured to emit light at a second, different wavelength;
a photodetector configured to measure intensities of light reflected from the patient at the first and second wavelengths; and
at least one electronic processor programmed to:
continuously estimate a bilirubin level in the patient by comparing the measured intensity of light at the first and second wavelengths; and
generate an indication of whether the continuously estimated bilirubin level in the patient has decreased to a safe level.

13. The device of claim 12, further including:
a display screen disposed on the housing;
wherein the at least one electronic processor is further programmed to control the display screen to output the generated indication as a displayed message.

14. The device of claim 12, further including:
a display screen disposed on the housing;
wherein the at least one electronic processor is further programmed to control the display screen to output the continuously estimated bilirubin level.

15. The device of claim 12, further including:
an alert light disposed on a portion of the housing;
wherein the at least one electronic processor further programmed to control the alert light to output the generated indication by illuminating the alert light.

16. The device of claim 12, wherein the at least one electronic processor is further programmed to:
control a phototherapy device to stop emitting phototherapy light or adjust a rate at which phototherapy light is emitted when the estimated bilirubin level has decreased to the safe level.

17. The device of claim 12, wherein the housing includes a top face, a bottom face, and a plurality of side faces disposed between the top and bottom faces, and the housing is configured for attachment to a patient with the bottom face contacting the patient;
wherein the at least two illuminators and the photodetector are mounted in at least one of the side faces.

18. The device of claim 12, wherein the at least one electronic processor is further programmed to:
apply a linear model to the continuously estimated bilirubin level in the patient to determine a remaining amount of time for phototherapy, the model further receiving as inputs at least patient age, patient skin color, and the safe level.

19. The device of, wherein the at least one electronic processor is further programmed to:
calibrate the photodetector with bilirubin levels obtained from blood of the patient.

20. A method of monitoring phototherapy delivered to a patient, the method comprising:
with at least two illuminators secured to a housing attached to the patient, emitting light towards at least a portion of the patient at a first wavelength and a second, different wavelength;
with at least two photodetectors, measuring intensities of light reflected from the patient at the first and second wavelengths; and
with at least one electronic processor:
continuously estimate a bilirubin level in the patient by comparing the measured intensity of light at the first and second wavelengths;
apply a linear model to the continuously estimated bilirubin level in the patient to determine a remaining amount of time for phototherapy, the model further receiving as inputs at least patient age, patient skin color, and the safe level; and
generate an indication of whether the continuously estimated bilirubin level in the patient has decreased to a safe level based on the continuously estimated bilirubin level.
